(19) United States
(12) Patent Application Publication
(10) Pub. No.: US 2023/0310780 A1
(43) Pub. Date: Oct. 5, 2023

(54) TRACHEAL CANNULA

(71) Applicant: Andreas Fahl Medizintechnik - Vertrieb GmbH, Koln (DE)

(72) Inventor: Andreas Fahl, Köln (DE)

(21) Appl. No.: 18/073,709

(22) Filed: Feb. 21, 2023

Related U.S. Application Data
(63) Continuation of application No. PCT/EP2021/066361, filed on Jun. 17, 2021.

(30) Foreign Application Priority Data
Jun. 17, 2020 (DE) 102020115938.2

(51) Int. Cl. A61M 16/04

(52) U.S. Cl. CPC A61M 16/0497; A61M 16/0465

(57) ABSTRACT

A tracheal cannula for laryngectomized and/or tracheotomized persons includes a cannula tube part, a connector part and at least two supporting wings for fastening a strap. The supporting wings are arranged on the connector part, wherein the connector part includes a first conical part and a second conical part. The first conical part is disposed on the cannula tube part and widens away from the cannula tube part. The second conical part is disposed on the first conical part and widens toward the first conical part.

### CROSS REFERENCE TO RELATED APPLICATIONS

This application is a continuation of PCT/EP2021/066361 filed Jun. 17, 2021 which claims priority of German Application No. DE102020115982.2 filed Jun. 17, 2020.

### BACKGROUND OF THE INVENTION

The invention relates to a tracheal cannula for laryngectomized and/or tracheotomized persons comprising a cannula tube part, a connector part and at least two supporting wings for fastening a strap.

Tracheal cannulas are generally known from the prior art. For example, DE 10 2009 013 424 A1 describes a tracheal cannula with a horizontal perforated screen.

The tracheal cannulae known from the prior art have the disadvantage that they do not seal well, especially with certain tracheostoma shapes.

### SUMMARY OF THE INVENTION

It is therefore the object of the invention to provide an improved tracheal cannula which in particular overcomes the disadvantages known from the prior art. In particular, it is the object of the invention to provide a tracheal cannula with improved wearing comfort. In particular, the object of the invention is to provide a tracheal cannula with an improved seal to the tissue surrounding the tracheostoma.

The object is solved according to the invention by means of a tracheal cannula for laryngectomized and/or tracheotomized persons comprising a cannula tube part, a connector part and at least two supporting wings for fastening a strap, wherein the connector part and cannula tube part are formed in one piece, wherein the supporting wings are arranged on the connector part, wherein the connector part comprises a first conical part and a second conical part, wherein the first conical part is disposed on the cannula tube part and widens away from the cannula tube part, and wherein the second conical part is disposed on the first conical part and widens towards the first conical part.

A tracheal cannula for laryngectomized and/or tracheotomized persons is proposed. This comprises a cannula tube part, a connector part and at least two supporting wings for fastening a strap, wherein the connector part and cannula tube part are formed in one piece. The supporting wings are arranged on the connector part, wherein the connector part comprises a first conical part and a second conical part. The first conical part is disposed on the cannula tube part and widens away from the cannula tube part. The second conical part is disposed on the first conical part and widens towards the first conical part.

Preferably, the first conical part tapers towards the cannula tube part. Further preferably, a smallest diameter of the first conical part is approximately equal to a diameter of the tracheal cannula, preferably a diameter of the tracheal cannula at its end facing the first conical part.

If, in the context of the invention, the term “about” is used in connection with values or ranges of values, this is to be understood as a range of tolerance which the person skilled in the art considers to be usual in this field, in particular a range of tolerance of ±20 %, preferably ± 10 %, further preferably ±5 % is provided.

Insofar as different value ranges, for example preferred and further preferred value ranges, are indicated in the present invention, the lower limits and the upper limits of the different value ranges can be combined with each other.

Preferably, the second conical part tapers away from the first conical part. Further preferably, a largest diameter of the second conical part is approximately equal to a largest diameter of the first conical part, which is further preferably immediately adjacent to the second conical part.

In the sense of the invention, a diameter is an inner diameter and/or an outer diameter, particularly preferably an outer diameter. Preferably, a smallest outer diameter of the first conical part is approximately equal to an outer diameter of the tracheal cannula, preferably an outer diameter of the tracheal cannula at its end facing the first conical part. Further preferably, a largest outer diameter of the second conical part is approximately equal to a largest outer diameter of the first conical part, which is further preferably immediately adjacent to the second conical part.

The tracheal cannula comprises a cannula tube part. In one embodiment, the cannula tube portion is fenestrated or screened. In another embodiment, the cannula tube part comprises a horizontal screening. In another embodiment, the cannula tube part is unfenestrated and unscreened.

Preferably, a length of the cannula tube part is about 36 mm to about 55 mm. Preferably, the length of the cannula tube part is a distance from the connector part to the proximal end opposite the connector part.

If directional indications are used in the description of the invention, they are to be understood with reference to the usual use of the tracheal cannula. Directional designations are used as is customary in anatomy. In particular, medical direction indications can be used even if the tracheal cannula is not worn by a user. Here, the usual use of the tracheal cannula is assumed to determine the direction. The term “distal” is understood in the sense of the present invention with respect to a feature of the device according to the invention to mean an arrangement or use thereof remote from or also facing away from or opposite the tracheal cannula, more generally assignable to a skin surface of a person wearing the tracheal cannula in particular. By distal is preferably meant a direction away from the body of the user, in the intended use of the tracheal cannula. By the term “proximal” is understood, in the sense of the present invention, with respect to a feature of the device according to the invention, an arrangement or use thereof close to or also facing or adjacent to a tracheal cannula, more generally assignable to the center of the body of a person wearing the tracheal cannula. Proximal preferably means a direction towards the center of the user’s body when the tracheal cannula is intended to be used. For example, the cannula tube part extends distally from the connector part and caudally - towards the buttocks. For example, the connector part is arranged distally from the cannula tube part.

Preferably, an outer diameter of the cannula tube part is about 8 to about 12 mm, preferably about 8 mm, more preferably about 9 mm, more preferably about 10 mm, more preferably about 12 mm.

In one embodiment, the cannula tube part is made of a material selected from a group comprising at least silicone and/or TPE. In a further embodiment, it is provided that at least the cannula tube part (20) comprises a hardness of about 60 Shore-A to about 80 Shore-A according to DIN ISO 7619-1 (2012-02). Preferably, it is provided that the cannula tube part comprises a hardness of about 70 Shore-A.

The tracheal cannula further comprises a connector part. In one embodiment, the connector part is made of a material selected from a group comprising at least silicone and/or TPE. In one embodiment, the connector part comprises the same material as the cannula tube part. In another embodiment, the connector part has a different material than the cannula tube part. According to the invention the connector part and the cannula tube part are formed in one piece, preferably injection molded in one production step.

In a further embodiment, it is provided that at least the connector part comprises a hardness of about 60 Shore-A to about 80 Shore-A according to DIN ISO 7619-1 (2012-02). Preferably, it is provided that the connector part comprises a hardness of about 70 Shore-A. In one embodiment, it is provided that the connector part comprises a different hardness than the cannula tube part, in particular is harder than the cannula tube part. In another embodiment, the cannula tube part and the connector part comprise approximately the same hardness.

The connector part comprises the first conical part. In one embodiment, the first conical part comprises a first half opening angle. The first half opening angle is preferably about 20° to about 45°, more preferably about 38°.

In the sense of the invention, the half opening angle is an angle between the generatrices of an outer envelope, that is, the connecting distances of the points of a base circle of the first or second conical part with a tip of the respective conical part, and a vertical axis of the first or second conical part, respectively.

Preferably, the first conical part comprises a continuous recess, in particular from proximal to distal, further preferably from the side facing the first cannula tube part to the opposite side facing the second conical part. Further preferably, the first conical part is communicatively connected to the cannula tube part. In one embodiment, the recess is bounded by a preferably substantially conical inner wall of the first conical part.

The term “ substantially ” indicates a range of tolerance which is acceptable to the person skilled in the art from an economic and technical point of view, so that the corresponding feature is still recognizable or realized as such.

In one embodiment, the first conical part has a first material thickness. Preferably, the first material thickness is at least in sections approximately equal to a material thickness of the cannula tube part. Further preferably, the material thickness of the cannula tube part is about 1 mm to about 1.5 mm. Further preferably, the material thickness of the first conical part is, at least in sections, about 1 mm to about 1.5 mm.

The connector part comprises the second conical part. In one embodiment, the second conical part comprises a second half opening angle. The second half opening angle is preferably about 10° to about 45°, more preferably about 16°.

Preferably, the second conical part comprises a through recess preferably from proximal to distal, more preferably from the side facing the first conical part to the opposite side. Further preferably, the second conical part is communicatively connected to the first conical part. In one embodiment, the recess is bounded by a preferably substantially conical inner wall of the second conical part. In a further embodiment, the inner wall of the second conical part comprises a connector profile for a tracheostomy appliance. Preferably, the connector profile comprises a connector opening diameter of about 22 mm. Preferably, the second conical part forms a standard receptacle for tracheostomy aids. Further preferably, the connector profile is designed to accommodate a tracheostomy aid with a diameter of approximately 22 mm. Tracheostomy aids are, for example, heat-moisture exchangers, speaking valves and/or ventilators.

Exemplary enumerations are not to be regarded as conclusive in the sense of the invention, but may be supplemented within the scope of general technical knowledge.

In one embodiment, the second conical part has a second material thickness. Preferably, the second material thickness is at least in sections equal to the material thickness of the first conical part. Further preferably, the material thickness of the second conical part is at least in sections about 1 mm to about 2.5 mm, further preferably about 1.5 mm to about 2.5 mm.

In the sense of the invention, a cone is a geometrical body in which all points of a base surface are connected in a straight line with a point or a point of a plane in which the base surface is not located. The base surface may be circular, rectangular, oval or any other shape, in particular with rounded corners. Furthermore, in the sense of the invention, the recess of the first and/or the second conical part preferably has a circular conical configuration. In the sense of the invention, a cone can also be understood as a truncated cone. In particular, it is provided that the recess overlaps the first and/or second conical part in such a way that the first and/or second conical part is formed as a truncated cone. In one embodiment, a longitudinal axis of the recess is preferably congruent with the vertical axis of the first and/or second conical part, which is a perpendicular from a theoretical not formed tip to the base of the cone shape of the first and/or second conical part.

In one embodiment, the first conical part and the second conical part are provided adjacent to each other via a transition region. Preferably, a radius is formed in the transition region, preferably on an outer surface of the connector part, between the first conical part and the second conical part. Further preferably, the transition region has a transition point. The transition point is formed as an inflection point preferably of the outer surface of the transition region as viewed in a longitudinal section through the tracheal cannula. In an embodiment in which the first conical part and the second conical part are immediately adjacent to each other, and preferably the connector does not include a transition region, the transition region is the substantially discontinuous location on the outer surface where the first conical part and the second conical part are adjacent to each other. Preferably, the transition region preferably comprises a radius of about 3 mm to about 10 mm on the outer surface, further preferably about 5 mm. In one embodiment, the connector part has a maximum diameter, in particular a maximum outer diameter, at the transition point.

Preferably, the transition point is located on a frontal plane. Preferably, the frontal plane is the plane that is expected to be at least tangent to a skin surface of a user of the tracheostomy cannula when the tracheostomy cannula is disposed in the user’s tracheostoma.

The tracheal cannula comprises supporting wings. The supporting wings advantageously replace a cannula shield that completely surrounds the connector part and are thus much more comfortable and visually appealing than tracheal cannulae known from the prior art. In one embodiment, the supporting wings are arranged substantially in the transition region. Preferably, the supporting wings are arranged laterally on the connector part. Further preferably, the connector part preferably comprises a connection point in the transition region at which the supporting wings are arranged. Preferably, the supporting wings are materially bonded to the connector part. In a further embodiment, it is provided that the connector part has a greater material thickness at least in the region of the connection point than in other regions of the connector part. In a further embodiment, the material thickness of the second conical part is thicker than that of the first conical part. In a further embodiment, the connection point or the supporting wings are disposed only on the second conical part. Further preferably, the connection point is arranged distal to the transition point.

The supporting wings are designed to accommodate a strap by means of which the tracheal cannula can be held on the neck. In a further preferred embodiment, it is provided that the supporting wings each have at least one strap recess. In a further embodiment, it is provided that the strap recesses are preferably designed to be completely continuous through the supporting wings. Preferably, a width and/or width of the strap recess is adapted to the strap likely to be used, preferably such that the strap has substantially no play to move. This advantageously prevents movements of the strap, in particular to prevent chafing on the skin.

In one embodiment, the supporting wings extend laterally from the connection point. Further preferably, the supporting wings extend laterally and proximally from the connection point. In one embodiment, the supporting wings are curved. Preferably, these are curved or further preferably have a radius. Preferably, the wings are curved in the direction of the cannula tube part.

In another embodiment, it is provided that a radius of a curve of the supporting wings can be arranged substantially in a transverse plane. The transverse plane corresponds to the horizontal plane of a standing user.

In one embodiment, the supporting wings extend distally and laterally from a connection point on the connector part. Preferably, the supporting wings are directed more laterally in the area of the connection point and more distally in the area of a laterally located end of the supporting wings. Further preferably, a vector of distal extension becomes larger over the length of the supporting wing starting from the connection point. Further preferably, a vector of the extension to the lateral direction becomes smaller over the length of the supporting wing starting from the connection point.

In one embodiment, it is provided that an end of the supporting wings facing away from the connection point comprises a distance from a frontal plane on which a transition point arranged in the transition region is arranged. This embodiment advantageously prevents the supporting wings from contacting the skin surface of the user. Further preferably, in one embodiment, it is proposed that the distance is approximately equal to a width of at least one strap recess disposed in the supporting wings. Preferably, the spacing is equal to a thickness of a provided strap. In this way, it is ensured that the strap is at least partially congruent with the frontal plane with its side facing the cannula tube part, further preferably with its proximal side. Further preferably, this design ensures that the strap lies flat on the user’s skin surface and does not cause any pressure points.

Preferably, a distance of the connection point to the frontal plane of the transition point or the turning area of the transition point is provided to be approximately equal to a width of the strap recess, more preferably of a provided strap that can be used with the tracheal cannula. In one embodiment, the distance corresponds approximately to a width of at least one strap recess arranged in the supporting wings. A further preferred distance between the connection point and the frontal plane of the transition point or the turning area of the transition point is about 1 mm to about 5 mm, preferably about 2 mm.

Preferably, the strap recess is arranged in the area of a turning point of the respective supporting wing.

In a further embodiment, it is provided that the tracheal cannula comprises a marking. In a further embodiment, it is provided that the marking identifies a size and/or a length of the tracheal cannula. Preferably, the marking is disposed on an outer surface of a connector part. A marking is also preferably arranged on the second conical part. The arrangement of the marking on the second conical part has the advantage that the tracheal cannula does not need to be touched to determine the size of the tracheal cannula during normal storage. In particular, the conical shape of the second conical part means that the marking is also visible in the packaging, as it is not covered by surrounding packaging material.

An exemplary embodiment of the tracheal cannula includes a cannula tube part and a connector part. The connector part is materially bonded to the cannula tube part. The connector part comprises a first conical part and a second conical part. The connector part further comprises two supporting wings extending substantially laterally from a connection point. The supporting wings each have a strap recess through which a strap can be guided. A width of the strap recesses is designed in each case in such a way that it corresponds approximately to a thickness of the strap. The width of the strap recess is also designed in such a way that it corresponds approximately to a width of the strap.

The second conical part has a continuous second recess that communicates with a first recess of the first conical part, which in turn communicates with the cannula tube part. Further, the second conical part has an inner wall that includes a connector profile. By means of the connector profile, a tracheostomy aid not shown here, for example a moisture-heat exchanger, can be attached to the tracheal cannula. A marking is applied to the second conical part, which is clearly visible from the front due to the conical shape, and which preferably shows at least one size of the tracheal cannula.

The connector part, respectively the first conical part and the second conical part as well as the cannula tube part are designed in the exemplary embodiment to be materially bonded. In particular, the tracheal cannula is made of a silicone with a hardness of about 70 Shore-A. A transition region is formed between the first conical part and the second conical part, the transition region having an exemplary radius. A turning point of the transition region marks a transition point between the first conical part and the second conical part, which at the same time preferably represents a maximum penetration depth of the tracheal cannula into the tracheostoma of a user. The frontal plane on which the transition point is arranged is preferably at least partially the plane of the user’s skin surface when the tracheostomy tube is inserted into the tracheostoma. The supporting wings are arranged substantially distal to the frontal plane so that skin irritation and pressure points caused by the supporting wings can be avoided.

The first conical part has an exemplary first half opening angle between the vertical axis and a generatrix of an outer envelope of the first conical part of about 38°. The second conical part, whose base surface is associated with the base surface of the first conical part, has an exemplary second half opening angle of about 16° between the vertical axis and a generatrix of an outer envelope of the second conical part. In the exemplary embodiment, the vertical axis of the first conical part and the vertical axis of the second conical part and the longitudinal axes of the first recess and the second recess are identical. This arrangement of the conical parts is visually appealing and particularly comfortable when wearing the tracheal cannula.

Exemplarily, the second conical part comprises a second material thickness that is greater than a first material thickness of the first conical part. The second conical part comprises connector profile and is advantageously reinforced to hold the supporting wings without substantially deforming the connector part during a use. On the other hand, the first material thickness is designed to allow slight deformation to conform to the tissue surrounding the tracheostoma.

Further advantageous embodiments are shown in the following drawings. However, the further embodiments shown there are not to be interpreted restrictively; rather, the features described there can be combined with one another and with the features described above to form further embodiments. Furthermore, it should be noted that the reference signs given in the figure description do not limit the scope of protection of the present invention, but merely refer to the embodiments shown in the drawings. Identical parts or parts having the same function have the same reference signs in the following. Showing:

### BRIEF DESCRIPTION OF THE DRAWINGS

FIG. 1 depicts a tracheal cannula in a first view;

FIG. 2 depicts the tracheal cannula of FIG. 1 in a side view; and

FIG. 3 depicts the tracheal cannula of FIG. 1 in a top view.

### DETAILED DESCRIPTION

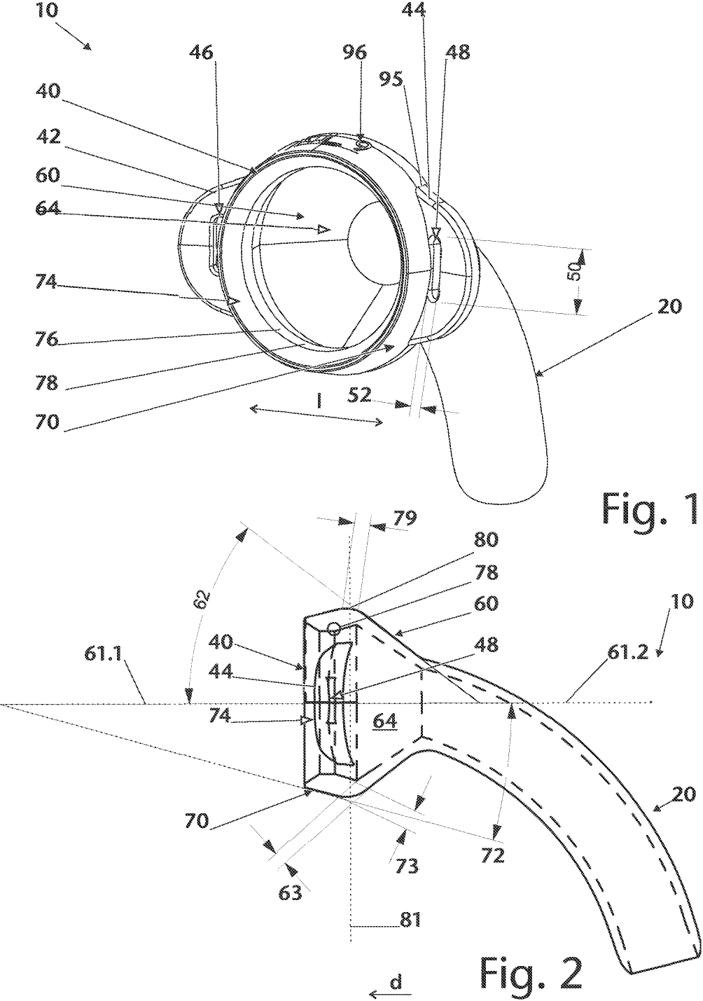

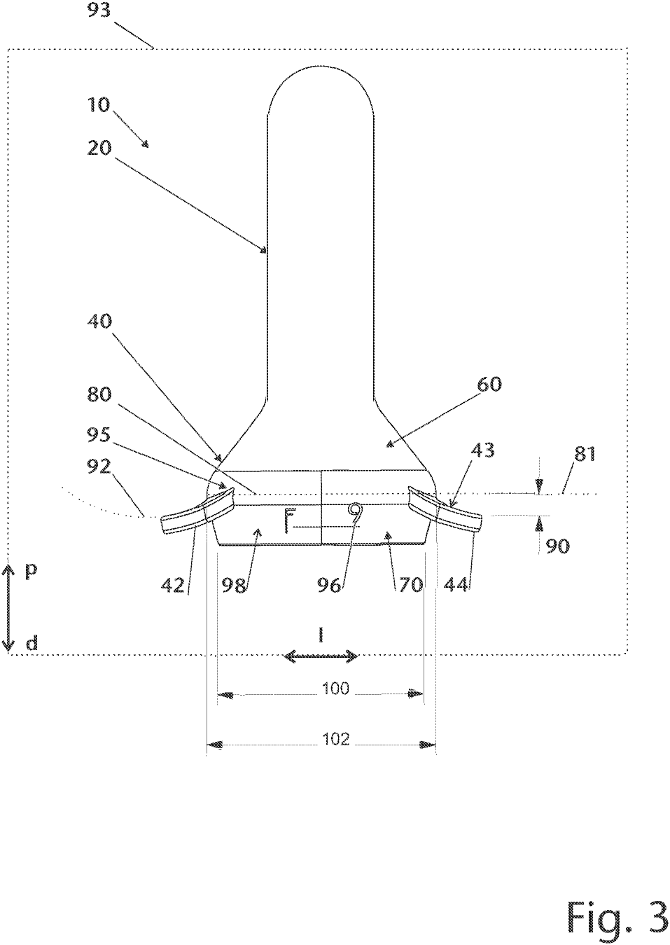

FIG. 1 shows a tracheal cannula 10 comprising a cannula tube part 20 and a connector part 40. The connector part 40 is materially bonded to the cannula tube part 20. The cannula tube part 20 includes a first conical part 60 and a second conical part 70. Two supporting wings 42, 44 are further disposed on the connector part 40 and extend substantially laterally 1 from a connection point 95. The supporting wings 42, 44 each have a strap recess 46, 48 through which a strap not shown here can be guided. A width 52 of the strap recesses 46, 48 is designed in each case in such a way that this corresponds approximately to a thickness of the strap. The width 50 of the strap recess 46, 48 is also designed in such a way that it corresponds approximately to a width of the strap.

The second conical part 70 includes a continuous second recess 74 that communicates with a first recess 64 of the first conical part, which in turn communicates with the cannula tube part 20. Further, the second conical part 70 has an inner wall 76 that includes a connector profile 78. By means of the connector profile 78, a tracheostomy aid not shown here, for example a moisture-heat exchanger, can be attached to the tracheal cannula 10. A marking 96 is applied to the second conical part 70, which is clearly visible from the front due to the conical shape, and which preferably shows at least one size of the tracheal cannula 10.

FIG. 2 shows the tracheal cannula 10 in a side view, with hidden edges indicated by dashed lines. The connector part 40, respectively the first conical part 60 and the second conical part 70 as well as the cannula tube part 20 are made of composite materials. In particular, the tracheal cannula is made of a silicone having a hardness of about 70 Shore-A. A transition region 79 is formed between the first conical part 60 and the second conical part 70, which has a radius that is not further described here. A turning point of the transition region 79 marks a transition point 80, which at the same time preferably represents a maximum penetration depth of the tracheal cannula 10 into the tracheostoma of a user. The frontal plane 81, on which the transition point 89 is arranged, is preferably at least partially the plane, of the skin surface of the user when the tracheal cannula 10 is inserted into the tracheostoma. The supporting wings 42, 44 are disposed substantially distal d of the frontal plane so that skin irritation and pressure points from the supporting wings 42, 44 can be avoided.

The first conical part 60 has a first half opening angle 62 between a vertical axis 61.1 of the first conical part 60 and an undesignated generatrix of an outer envelope of the first conical part 60 of about 38°. The second conical part 70, whose base surface not shown here is associated with the base surface of the first conical part 60 not shown, has a second half opening angle 72 of about 16° between a vertical axis 61.2 of the second conical part 70 and an unspecified generatrix of an outer envelope of the second conical part 70. In the embodiment shown in FIG. 2, the vertical axis 61.1 of the first conical part 60 and the vertical axis 62.2 of the second conical part 70 and the longitudinal axes of the first recess 64 and the second recess 74, which are not further specified, are identical. This arrangement of the conical parts 60 and 70 is visually appealing and particularly comfortable when wearing the tracheal cannula.

The second conical part 70 comprises a second material thickness 73 that is greater than a first material thickness 63 of the first conical part 60. The second conical part 70 comprises the connector profile 78 referred to in FIG. 1 and is advantageously reinforced to hold the supporting wings 42, 44 without substantially deforming the connector part 40 during use. In contrast, the first material thickness 63 is designed to allow slight deformation to conform to the tissue surrounding the tracheostoma.

The proposed tracheostomy cannula 10 can be worn comfortably and avoids pressure points and skin irritation due to its geometry.

1. Tracheal cannula for laryngectomized and/or tracheotomized persons comprising:
a cannula tube part, a connector partand at least two supporting wingsfor fastening a strap, wherein the supporting wingsare disposed on the connector part, wherein the connector part comprises a first conical part and a second conical part, wherein the first conical part is disposed on the cannula tube part and widens away from the cannula tube part, and wherein the second conical part is disposed on the first conical part and widens towards the first conical part.

2. Tracheal cannula according to claim 1, wherein the first conical part and the second conical part are adjacent to each other via a transition region.

3. Tracheal cannula (10) according to claim 2, wherein the supporting wings are disposed substantially in the transition region.

4. Tracheal cannula according to claim 1 the supporting wings are curved.

5. Tracheal cannula according to claim 4, wherein a radius of a curve of the supporting wings can be disposed substantially in a transverse plane.

6. Tracheal cannula according to claim 1 wherein the supporting wings extend distally (d) and laterally (1) from a connection point on the connector part (40).

7. Tracheal cannula according to claim 6, wherein an end of the supporting wings facing away from the connection point comprises a distance to a frontal plane on which a transition point arranged in the transition region is arranged.

8. Tracheal cannula according to claim 7, wherein the distance corresponds approximately to a width of at least one strap recess arranged in the supporting wings.

9. Tracheal cannula according to claim 1 wherein the cannula tube part comprises a hardness of about 60 Shore-A to about 80 Shore-A according to DIN ISO 7619-1 (2012-02).
